(19) United States
(12) Patent Application Publication
(10) Pub. No.: US 2018/0039750 A1
(43) Pub. Date: Feb. 8, 2018

(54) PROCESSING REPEATED ERROR MESSAGES IN A DIALYSIS MACHINE

(71) Applicant: Fresenius Medical Care Deutschland GmbH, Bad Homburg (DE)

(72) Inventor: Alexander Schrörs, Frankfurt (DE)

(21) Appl. No.: 15/657,834

(22) Filed: Jul. 24, 2017

(30) Foreign Application Priority Data
Aug. 5, 2016 (DE) 102016114539.4

(51) Int. Cl. G06F 19/00; A61M 1/16

(52) U.S. Cl. CPC G06F 19/3412; A61M 1/1601; A61M 2205/50; A61M 2205/505; A61M 2205/52

(57) ABSTRACT

The present invention relates to an error processing unit for a medical machine (e.g., a hemodialysis machine or a peritoneal dialysis machine). The error processing unit is configured to detect a number of consecutive confirmation inputs in response to each currently output machine error message, representing a status—requiring a message—of the dialysis machine, as an ACTUAL value. When a preconfigured TARGET value is exceeded by the ACTUAL value, a modified machine control is triggered and results in additional or modified outputs being output on a graphical user interface.

### CROSS-REFERENCE TO RELATED APPLICATIONS

Priority is claimed to German Patent Application No. DE 102016114539.4, filed on Aug. 5, 2016, the entire disclosure of which is hereby incorporated by reference herein.

### FIELD

The present invention relates to error processing in dialysis machines or other medical-technology machines. It relates in particular to an error processing unit, to a medical machine having an error processing unit, and to a method for operating a medical machine when processing messages.

### BACKGROUND

Current, modern, medical-technology machines, such as for example the hemodialysis system 5008 from Fresenius Medical Care, are generally equipped with a touch-sensitive screen, a touchscreen, for controlling and operating the machine and for inputting and outputting messages.

In clinical practice, the error-free functioning of the medical-technology machine is important. Therefore, a multiplicity of detectors (e.g., sensors) are incorporated in the machine and detect a status of the machine and inform the user of the status, as appropriate, via a message via the user interface. These messages can signal, for example, an error status on the machine. The messages should be acknowledged by the user. In the event of an error message, generally an action must be performed on the machine in order to return the status to an error-free status. If the message relates for example to the instruction that the cover doors on the machine are open, then these must first be closed in order for the machine to then be able to be operated. The user will thus initially confirm or acknowledge the message in order then to close the doors. This is detected by the machine, and so the message no longer appears. However, if the user does not close the doors and thus does not change the error status, the message is repeatedly output.

In the prior art, the process of <error status—message—message acknowledgement—no error correction on the machine> occurred with arbitrary frequency and was iteratively performed unaltered, always with the same message. Therefore, in a disadvantageous manner, there is the risk that the error status lasts for too long a time because the user does not know which error correction measures on the machine he actually has to perform in order to correct the error status. This process is not productive. The problem in the known machines from the prior art is that occasionally too much time passes before an error status is corrected which can lead to a time delay for the medical treatment.

### SUMMARY

In an exemplary embodiment, the present invention provides a dialysis machine. The dialysis machine includes a processor and a non-transitory computer-readable medium, the non-transitory computer-readable medium having processor-executable instructions stored thereon, wherein the processor-executable instructions, when executed by the processor, facilitate performance of the following: outputting a number of consecutive machine error messages; and determining a number of consecutive confirmation inputs received in response to the consecutive machine error messages as an ACTUAL value.

In another exemplary embodiment, the present invention provides a method for operating a dialysis machine. The method includes: outputting, by the dialysis machine, a number of consecutive machine error messages; and determining, by the dialysis machine, a number of consecutive confirmation inputs received in response to the consecutive machine error messages as an ACTUAL value.

In yet another exemplary embodiment, the present invention provides a method for operating a dialysis machine. The method includes: determining, by the dialysis machine, an error status via sensors of the dialysis machine; outputting, by the dialysis machine, a machine error message in response to the determined error status; receiving, by the dialysis machine, confirmation input via a user interface of the dialysis machine and determining whether the error status has been addressed; and based on the machine error message being output a predetermined consecutive number of times without the error status being addressed, initiating, by the dialysis machine, modified machine operation.

### BRIEF DESCRIPTION OF THE FIGURES

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a schematic illustration of a dialysis machine having an error processing unit in accordance with an embodiment of the invention;

FIG. 2 shows an example of a flow diagram for an operating method of a dialysis machine according to an embodiment of the invention;

FIG. 3 shows a schematic illustration, in table format, of machine statuses and a change, in accordance with an embodiment of the invention, of an ACTUAL value during operation of a dialysis machine;

FIG. 4 shows an exemplary schematic illustration of a data exchange of signals between an error processing unit and a user interface of the dialysis machine according to an embodiment of the invention.

### DETAILED DESCRIPTION

Exemplary embodiments of the invention improve the processing of dialysis machine messages and reduce the time frame for reaching an error-free operating status. Furthermore, the process for dialysis machine operation is improved in terms of dialysis machine messages, which in turn increases the quality of the use of the machine.

The invention will be described hereinafter with the aid of the solution to the problem in terms of the device, and thus inter alia with the aid of the error processing unit. Features, advantages or alternative embodiments mentioned in this respect can likewise be transferred to the other claimed subject matter, and vice versa. In other words, the claims relating to other claim categories (which are directed claims categories e.g. to a system or to a device) can be developed with the features which are described or claimed in conjunction with the method. The corresponding functional features of the method are thereby embodied by corresponding physical modules, in particular by electronic hardware modules or microprocessor modules of the system or of the product, and vice versa.

Additionally, it will be appreciated by those of ordinary skill in the art that the execution of the various machine-implemented processes and steps described herein may occur via the computerized execution of processor-executable instructions stored on a non-transitory computer-readable medium, e.g., random access memory (RAM), read-only memory (ROM), programmable read-only memory (PROM), volatile, nonvolatile, or other electronic memory mechanism. Thus, for example, the operations described herein performed by computing devices and components thereof may be carried out according to stored instructions and/or installed applications.

According to a first aspect, the invention relates to an error processing unit or generally to a message processing unit for a medical-technology machine (e.g., a dialysis machine). The error processing unit or message processing unit is configured to detect a number of consecutive confirmation inputs in response to each currently output machine message as an ACTUAL value. The machine message can be a machine error message. For the sake of simplicity, the terms “error processing unit” and “machine error message” are used hereinafter.

The error processing unit is an electronic component. It can be embodied in hardware and/or software and is used to process messages of the machine—for example, error messages or status messages which are output on a user interface of the machine (e.g. optically/graphically and/or acoustically). The messages relate to a status of the medical-technology machine and can be, in particular, error messages. The error processing unit may be allocated to a medical-technology machine in each case. The error processing unit can exchange data with a central server which collects and evaluates (e.g., via a statistical evaluation) data from all, or selected ones, of the error processing units (or other medical-technology machines, such as from all dialysis machines of a dialysis station). It is also possible to configure one error processing unit for a plurality of medical-technology machines which can then resolve the messages of the machines according to machine identity (e.g., via a corresponding identification notice). It is likewise possible to configure a plurality of error processing units for one and the same medical-technology machine. The respective error processing units can be responsible for different technical machine components.

Embodiments of the invention will be described hereinafter for a dialysis machine (e.g., a hemodialysis machine or a peritoneal dialysis machine) as an example of a medical-technology machine. It will be appreciated by one of ordinary skill in the art that exemplary embodiments of the invention may also include other medical-technology, computer-controlled machines or (fluid management) machines or blood treatment machines, which output machine messages or machine error messages via a user interface.

The confirmation input is an input by the user on the user interface of the machine. The confirmation input can be input on an input field (e.g., on a switching element of a graphical user interface (GUI)). The confirmation input occurs in response to a message on the machine, in particular a machine error message. In an embodiment, the confirmation input may be input on the input field only after a machine error message has been output. For instance, provision can be made for example that the input field for detecting the confirmation input is only displayed after a machine error message has been output, for which a confirmation input has not yet been detected. The confirmation input can be input, for example, on a field or button of a touchscreens. Alternatively, the confirmation input can also be detected as an acoustic signal or by pressing a switch on the machine or a switching element.

Consecutive confirmation inputs are confirmation inputs which are input in succession for the same message (different messages but with the same content). If, for example, an error message is output and the error status has not changed because no action has been performed on the machine, then the error status continues. In this case, after confirmation of the message by the confirmation input, the error message is repeatedly output and then needs to be confirmed again, and so on. The confirmation inputs each related to the same message are thus called “consecutive.” Confirmation inputs are consecutive when the status of the machine has not changed in the meantime. However, if a first message relates, for example, to a first error status and is confirmed or acknowledged by a first confirmation input and subsequently, after the first error status is corrected, a second message relating to a second error status is acknowledged by a second confirmation input, then the first and second confirmation inputs are not called “consecutive”.

In an embodiment of the invention, all the machine error messages and all the confirmation inputs are stored in a log file of a memory. Then, it can be configured such that a particular modified control procedure is triggered when machine error messages of the same content follow each other. For example, if a message “Fluid level is too low. Fill with fluid” is output and this message status is corrected by a corresponding machine control (fluid is replenished) and, within a configurable time frame thereafter, the same message is output, then this situation could be evaluated and indicate that possibly the fluid container is leaky. After this evaluation, a modified machine control is then initiated and, for example, an additional message with the corresponding indication (leakage) is output on a graphical user interface.

In an embodiment of the invention, a status requiring a message can be an error status of the medical-technology machine which can be changed or corrected as a result, in that a manipulation is carried out on the machine or a status change is carried out on the machine. In principle, two different statuses are differentiated in accordance with the invention: an error-free operating status (normal operation of the machine) which is free of messages, and a message status in which messages are output. Messages can be output, for example, for errors occurring on the machine, for indications for the user (e.g., low filling level of a resource, normal values for selected parameters being exceeded or failing to be reached, etc.), for alarms or for emergency signals. It can be preset that the machine is automatically transferred to the status requiring a message if the error-free operating status is no longer applicable.

An output machine error message may relate to an error status. It can, however, also relate to a status change of the machine without an error occurring on the machine (e.g., if the user is only being informed of a particular situation) and may thus actually be a machine message. However, with respect to exemplary embodiments of this invention both such machine messages and messages are referred to as machine error messages which require an acknowledgement from the user via a confirmation input.

The machine error message and the field for the confirmation input are output on the user interface of the medical-technology machine, for example, on a graphical interface such as a touchscreen (a monitor or display). Alternatively, the error processing unit can also have a dedicated user interface, via which the messages are output. Interaction areas, switching elements, control fields, etc. for controlling the dialysis machine can be displayed on the user interface (e.g. the touchscreen).

Exemplary embodiments of the invention provide that the number of repeatedly output machine error messages having the same content (which thus relate to the same status of the machine) or the number of consecutive confirmation inputs related thereto are detected and evaluated in order to initiate modified operation of the machine.

In an embodiment of the invention, the error processing unit comprises a memory in which a preconfigured TARGET value for the number of consecutive confirmation inputs is stored. The TARGET value indicates from which number of repeated machine error messages a modified machine control or a modified operation of the machine (e.g., with modified actuation of the input/output interface and in particular of fields/elements on the graphical user interface) is to be performed.

In an advantageous embodiment of the invention, a plurality of TARGET values can be configured which define different value ranges (e.g. the TARGET values 3, 5 and 7). Therefore, a first value range between the first and 3^{rd} repeated input of the confirmation signal and a second value range between the 3^{rd} and the 5^{th} repeated input and a third value range from the 7^{th} input are defined. A specific, configurable control procedure is allocated to each of the value ranges (three in the example). In a configuration phase of the method, it is possible to configure which actions and measures are to be triggered in the respective control procedures, such as displaying other or additional input and output fields on the graphical user interface. Therefore, the modified control of the machine can occur depending on in which value range the detected ACTUAL value is located. In principle, after a TARGET value is first reached, a first machine operation can be triggered and when a TARGET value is reached for the second time, a second machine operation can be triggered, etc. In the previously described example, it would thus be possible to configure that, in the case of a triple repetition, an additional indication is output and in the case of a five-fold repetition additionally or alternatively an alarm signal is output and in the case of a seven-fold repetition a message to a preconfigured entity (colleague or supervisor) is automatically effected.

For example, in the case of a preconfigured TARGET value of 10, it can be defined that in the case of a 10-fold “rejection” of an error message (without correcting the error on the machine) an escalation of the machine reaction is initiated. In contrast, this escalation would not occur when the error message is confirmed or acknowledged for the first time.

If the TARGET value is, for example, 3, then provision can be made that after triple repeated machine error messages and thus after triple repeated or consecutive confirmation inputs, another machine operation is initiated. For instance, it is possible to configure that the future machine error message includes an additional indication which instructs the user as to which precise steps he must perform on the machine and which instructions he must follow in order to be able to correct the error.

In order for the error processing unit to be able to compare the detected number with the stored TARGET value or to otherwise process the detected number (e.g., with a statistical method), the error processing unit can include an evaluation unit. The evaluation unit is intended to compare the detected ACTUAL value with the stored TARGET value(s) for coincidence and, if the TARGET value is exceeded, to initiate a modified machine control (for output automated or alternative messages or for triggering a modified machine control).

The error processing unit or the evaluation unit can be configured to evaluate whether, how often in which time frame and/or by which operating person the currently output machine error message has been confirmed by a confirmation input. The evaluation unit can be provided in the form of an electronic processing unit (e.g., a central processing unit (CPU) or CPU component) or as a software module. The output machine error messages, the detected confirmation inputs and/or the evaluations are stored in a LOG file, for example, for further statistical evaluations.

In an embodiment of the invention, the error processing unit includes an output unit. This can be formed as a separate dedicated monitor which is allocated to the error processing unit or it can be formed as a touch field on the monitor of the medical-technology machine. The output unit is used to output the detected ACTUAL value and, for example, the TARGET value.

In an advantageous development of the invention, the error processing unit is designed to reset the detected number of consecutive confirmation inputs if a change in the machine status and/or an error correction has been performed on the medical machine, and so the medical device is in a message-free operating status (without errors).

In a further aspect, the invention relates to a medical-technology machine and in particular to a dialysis machine, having an error processing unit as described above.

In yet a further aspect, the invention relates to a method for operating a medical-technology machine, in particular a dialysis machine. During the operation of the medical-technology machine, a number of consecutive confirmation inputs will be detected for each output machine error message in response to the currently output machine error message as an ACTUAL value.

In accordance with an advantageous embodiment, the method includes the following method steps:
comparing the detected ACTUAL value with at least one preconfigurable TARGET value for the number of consecutive confirmation inputs and, if the at least one TARGET value is exceeded in a preconfigurable time interval:
   controlling, in a modified manner, the medical-technology machine by automatically outputting an additional message or by outputting a modified message on the basis of the detected ACTUAL value.

In accordance with one variation, the machine error message and/or the confirmation input is output or detected on a monitor of the medical-technology machine, in particular on a touchscreen monitor.

In accordance with another advantageous embodiment, the ACTUAL value is automatically reset if a machine status (e.g. error status) causing the output machine error message no longer exists.

In accordance with another advantageous embodiment, a modified control of the medical machine can be activated and deactivated or can be triggered automatically after a predefinable event has occurred. This has the advantage that the normal message-confirmation-machine reaction cycle can be retained for training purposes or other particular application purposes.

In an advantageous variation, provision is made that for all the output machine error messages, information as to how many confirmation inputs have been input by which user is detected and/or stored. For the purposes of a statistical evaluation, the dataset can be itemized into different parameters (e.g., into time frame, identity of the user after successful authentication, type of message, etc.).

In another advantageous embodiment of the invention, the method includes the following:
detecting a user identity which is specific for each user of the medical-technology machine (e.g., via authentication) and
controlling, in a modified manner, the medical machine in response to the detected ACTUAL value and the detected user identity.

For instance, the alternative machine reaction triggered in accordance with the invention can also be fixed, in dependence upon the classification of the user. The classification in turn can be detected, for example, by a preceding authentication of the user at the dialysis machine. For this purpose, the user may be requested, prior to operating the machine, to input a PIN code or a smart-card identifying him is read. Alternatively, biometric data can also be allocated for authenticating the user. Accordingly, for example, trainees can be supplied with comprehensive support which is output on the graphical user interface when consecutive confirmation inputs are detected. The support can display instructions and specific operating steps as to how the error status, requiring a message, of the machine can be transferred to the error-free operating status.

In another advantageous embodiment of the invention, the TARGET value is configured in dependence upon the detected user identity. Therefore, the classification of the machine user can be incorporated directly into the determination of the TARGET value. This has the advantage that, for example, a trainee can be provided in good time with the necessary information for correcting a machine error, in that the TARGET value is set to be lower than for an experienced person.

Another exemplary embodiment of the invention provides a computer program product which is loaded or can be loaded in a memory of a computer or of an electronic or medical-technology machine, having a computer program for implementing the method described in more detail above when the computer program is run on the computer or the electronic or medical-technology machine.

Another exemplary embodiment of the invention provides a computer program for implementing all the method steps of the method described in more detail above when the computer program is run on a computer, an electronic or medical-technology machine. It is thereby also possible for the computer program to be stored on a medium which can be read by the computer or the electronic or medical-technology machine.

The following detailed description discuss, in a non-limiting manner, exemplary embodiments with the features and further advantages thereof with the aid of the drawing.

Exemplary embodiments of the invention will be described in more detail hereinafter in conjunction with the figures.

The invention relates to the processing of messages of a medical-technology machine, for example a dialysis machine D. During dialysis treatment of a patient, it is occasionally of life-saving importance to ensure that the dialysis machine D functions in an error-free manner. Therefore, disruptions and machine errors should be signaled immediately. In the event of a disruption to the dialysis machine D a message automatically-appears on a graphical user interface GUI of the dialysis machine D, via which the user is informed of the disruption or the error. The message can contain further instructions which provide the user with support in order to solve the problem forming the basis of the current message.

The user, upon machine error messages being output, may be tempted in the daily hustle and bustle of a clinic to simply remove the machine error message without effecting a specific status change on the dialysis machine D. In other words, there is the risk that an attempt is made to “reject” the machine error message without performing specific problem-treating measures on the dialysis machine D. Therefore, the problem remains and the corresponding machine error message should be repeatedly output. In the known systems from the prior art, this procedure was previously repeated multiple times without adapting the content to the machine error message. Exemplary embodiments of the present invention, however, detect a number of consecutive confirmation inputs and further process the detected number in an evaluation unit. Consecutive confirmation inputs are those confirmation inputs which the user inputs in relation to the same machine error message. This can relate to the second or a multiple repetition of a confirmation input in relation to the same machine error message if the disruption causing the respective machine error message has not been corrected and the dialysis machine D is still in a status requiring a message.

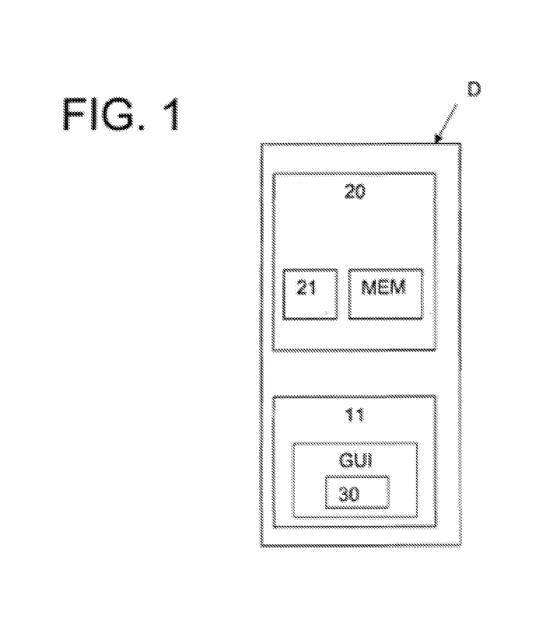

With reference to FIG. 1, exemplary components of the dialysis machine D in accordance with an embodiment will be explained in more detail hereinafter. The dialysis machine D includes, as a central unit, the error processing unit 20 which is configured to detect a number of consecutive confirmation inputs in response to each currently output machine error message, representing a status—requiring a message—of the dialysis machine D, as an ACTUAL value. If, for example, corresponding sensors which are arranged on the dialysis machine D or on the modules in interexchange with the dialysis machine D detect that a disruption or problem has occurred, such as for example a problem on the dialysis machine D or on the hose system, then this is signaled via the corresponding machine error message and is displayed to the user on the graphical user interface GUI. Thereupon, the user can press a button likewise displayed on the user interface GUI. This action is used as a confirmation input and relates to the machine error message overlaid immediately thereon and currently still displayed. If the user, in response, does not solve the problem on the machine or on the hose system, the error remains and after a preset time the same machine error message is output again. If, in turn, the user presses the corresponding field and inputs a confirmation input, this is a consecutive confirmation input which is detected in accordance with the invention. In accordance with the invention, the ACTUAL value count is incremented.

As further shown in FIG. 1, the dialysis machine D and in particular the error processing unit 20 can include a memory MEM in which a preconfigurable TARGET value for the (permitted) number of consecutive confirmation inputs is stored. The dialysis machine D and in particular the error processing unit 20 can further include an evaluation unit 21. The evaluation unit 21 is configured to compare the detected ACTUAL value with the stored TARGET value for coincidence. If the TARGET value is exceeded by the ACTUAL value, a modified machine control is triggered. The modified machine control can result in the outputting of alternative machine error messages or a modified machine control of the dialysis machine can be initiated. If the TARGET value is set, for example, to “3” then in one exemplified embodiment it is possible to configure that after a third consecutive confirmation input by the user is detected, a further (new) instruction field appears on the graphical user interface GUI showing further measures and references which instruct the user how be can solve the respective disruption or problem of the dialysis machine D. The modified machine control can include still further measures such as, for example, conveying a corresponding exceedance message to further computer-based entities. The error processing unit 20 can include an output unit 30 which is configured to display or output the detected ACTUAL value alone or in combination with each configured TARGET value. The evaluation unit 21 and the output unit 30 can be components of the error processing unit 20. As illustrated in FIG. 1, alternatively the evaluation unit 21 and/or the output unit 30 can also be separate modules which are arranged partly in the dialysis machine D. In an embodiment, the output unit 30 can be formed on a touchscreen monitor of the dialysis machine D. The dialysis machine D fundamentally includes a touchscreen monitor 11 which is intended to input/output data. In other words, at least one element is provided on the touchscreen monitor 11 on which particular values can be output (or input).

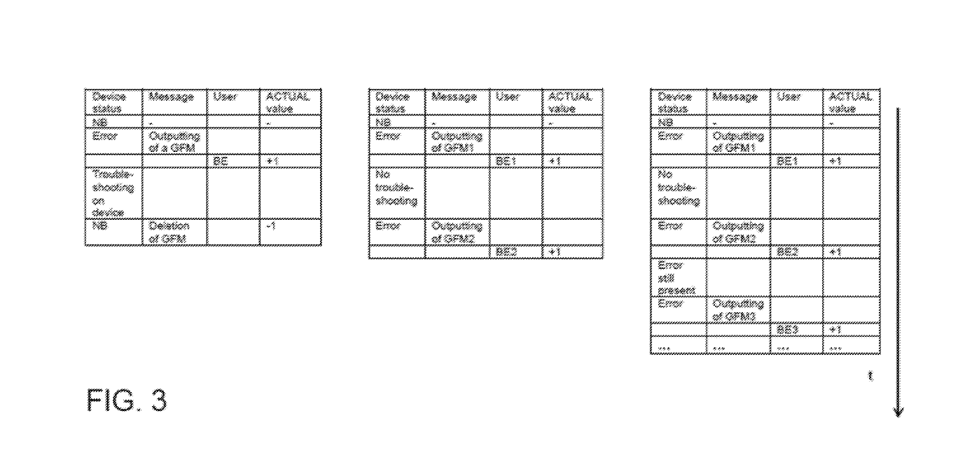

A typical process in accordance with an exemplary embodiment of the invention is explained in more detail hereinafter with reference to FIG. 3. FIG. 3 illustrates, by way of example, a plurality of different error processing routines and processes, in parallel with each other, for a dialysis machine D. As shown by the arrow on the right-hand side in FIG. 3, the time flows from the top to bottom.

In the left-hand table of FIG. 3, the dialysis machine D is initially operated in normal operation NB in which no messages are output and the user also does not have to effect any machine disruptions or problem treatments; the ACTUAL value is 0. After an error has been detected, a machine error message GFM is automatically output. Thereupon the user acknowledges the respective machine error message GFM by inputting a confirmation signal BE via a corresponding input field. In accordance with the invention, the ACTUAL value is incremented. If the user additionally also performs an error correction measure on the dialysis machine D and the problem can successfully be corrected (e.g., “troubleshooting on device”), the dialysis machine D can then be transferred to normal operation NB and the machine error message GEM may be deleted. No further machine error message is output and the ACTUAL value is then decremented or reset to zero. Therefore, the system is back in the initial status and can be monitored for further disruptions.

In the table shown in the center of FIG. 3, the same initial situation is represented. In this case, however, the user does not effect any error processing and the problem remains. After a first machine error message GFM1 has been output and acknowledged by the confirmation input BE1, subsequently a second machine error message GFM2 is output and acknowledged by a second confirmation input BE2. In this case, the ACTUAL value is incremented twice, and is, in this case, 2. Of course, the problem may ultimately be solved by the user and the dialysis machine D can then be transferred to normal operation NB. However, if this is not the case, then further machine error messages can be output.

On the right-hand table in FIG. 3, the case is illustrated in which the error remains even after outputting a second machine error message GFM2. In this case, a third machine error message GFM3 is output which is acknowledged in turn by a third confirmation input BE3. In this case, the ACTUAL value is likewise incremented and is, in this case, 3. If the comparison of the ACTUAL value with the TARGET value results in the TARGET value being exceeded, then, for example, an alarm message can be triggered. Furthermore, further actions can be initiated. The further actions can trigger for example the outputting of a corresponding warning message on the graphical user interface GUI of the dialysis machine D or on a server exchanging data with the dialysis machine D, namely to the extent that a confirmation input has already been input for the third time without the dialysis machine disruption having been corrected. For a person skilled in the art it is likewise within the scope of the invention to configure further measures and/or to provide further iterations. The machine error messages which are output in accordance with the invention can deviate from one another. It is therefore in particular possible for the second machine error message and third machine error message to each have a different content from the first machine error message. Furthermore, the subsequent machine error messages can provide additional instructions for the user as to how he can solve the specific problem. In particular, he can be provided with support as to which person he can contact or where he can find additional information.

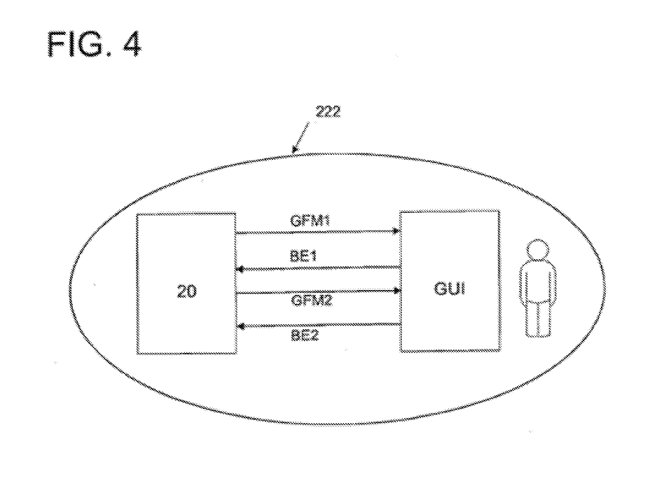

The operation of the error processing unit 20 in the event of an error will be explained in more detail hereinafter with reference to FIG. 4. If a status, requiring a message, in particular a disruption or error status 222, has been detected, then in accordance with the invention a plurality of signals are exchanged between the error processing unit 20 and the graphical user interface GUI which the user operates. After the error is detected, a first machine error message GFM1 is illustrated on the user interface GUI which the user can confirm via a confirmation input BE1. If the error continues and the problem is not solved and the dialysis machine D is thus still in a disruption or error status 222, then at a subsequent point it time, which is configurable, a second machine error message GFM2 is output on the interface GUI and can in turn be confirmed by the user by inputting a consecutive confirmation input BE2. After the second confirmation input BE2 has been detected, the ACTUAL value is set to 2 in accordance with the invention. In FIG. 4, merely two iterations of consecutive confirmation inputs are illustrated. Of course, a plurality of iterations can also be effected in this case. Each output machine error message GFM1, GFM2, . . . GFMi can be different from the others. The subsequent machine error messages fundamentally include further instructions for the user in order to support him in correcting the disruption forming the basis of the machine error message or in solving the problem.

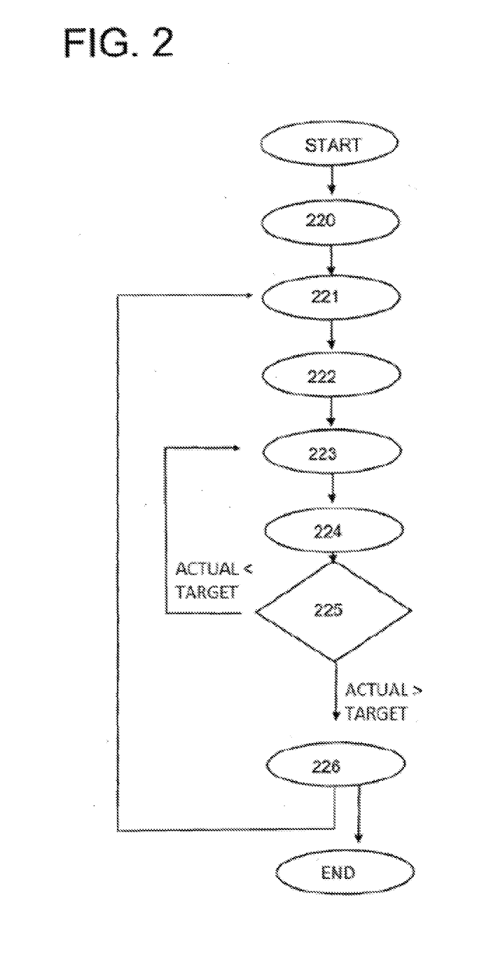

A process of the method in accordance with the invention in accordance with an embodiment will be explained in more detail hereinafter with reference to FIG. 2. After the method starts, in step 220 the user identity can be detected. This can be effected for example via an authentication or via inputting biometric data or a PIN. In step 221, the dialysis machine D can be operated in normal operation NB. In this status, there are no messages. In step 222, a status requiring a message is detected, for example a disruption of the dialysis machine. Thereupon, in step 223 a machine error message is output. Furthermore, in step 223 the output machine error message is acknowledged by inputting a confirmation input. In step 224, the ACTUAL value is detected. In this case, the counter for the confirmation inputs is thus previously incremented. In step 225 the currently detected ACTUAL value is compared with the preconfigured TARGET value. If the comparison determines that the ACTUAL value is below the TARGET value, a further machine error message (same content as the first) can be output, and the method returns to step 223. However, this is the case only when the problem forming the basis of the respective machine error message is not solved by the user. Otherwise, if the disruption is corrected, the ACTUAL value is decremented and step 221 can be continued with normal operation NB of the dialysis machine. If the comparison between the ACTUAL value and TARGET value shows that the ACTUAL value is above the TARGET value, then in accordance with the invention a modified machine control is automatically initiated. This is shown in FIG. 2 with reference sign 226. The modified machine control can include a modified outputting of a new additional machine error message or the machine error message can be supplemented with additional, further, extra instructions which characterize the respective current situation and the genesis or history of the origins thereof with the preceding error treatment. Furthermore, further measures and actions can be triggered. For instance, it is possible for example that the exceeding of the TARGET value is represented on the graphical user interface as a warning sign. Furthermore, the exceeding of the TARGET value can be forwarded as a dataset to other computer-based entities. Furthermore, an instruction can be output that this exceeding of the TARGET value is retained and stored in a memory and is thus available for statistical evaluations. Therefore, it can be rendered transparent for the user that his consecutive confirmation inputs are subjected to a statistical evaluation and storage.

The error processing unit 20 in accordance with the invention should automatically evaluate whether, and if so in which time frame and how frequently, the currently output machine error message has already been confirmed by the user. After a certain number of consecutive confirmation inputs per configurable unit of time, a preconfigured modified machine reaction 226 is automatically triggered. The modified machine reaction 226 may be a modified actuation of the user interface GUI or a modified screen reaction. The user may be informed by the modified machine reaction 226 that he should perform actions on the dialysis machine D in order for the machine status (e.g., the error status) to be able to be changed in order to be able to revert to the normal operation NB of the medical machine as quickly as possible. In other words, the user can therefore more quickly perform the necessary manipulations on the machine or on connected technical operating components, for example, on a hose set, for the benefit of the patient.

For the modified machine reaction 226, provision can be made that corresponding support text for the error message is displayed automatically (and not just after a corresponding confirmation by the user, for example, pressing a help button on the user interface GUI). It is also possible that an additional, separate message with different visuals is displayed which shows the user that preceding machine error messages have been confirmed with the number corresponding to the ACTUAL value without causing a machine reaction or without an effect on the dialysis machine D. Suggestions may then be made to the user as to how he can find additional information. In order to render the modified machine error message more noticeable to the user, it is also possible to retain the text itself but to position the output of the message at a different position on the user interface GUI or to format the message in a different manner (e.g., in a different color or in another font, font size and/or style). Furthermore, an additionally output alarm signal can be output alternatively or cumulatively. The alarm signal can be provided, for example, acoustically or also visually on an input/output interface.

Reference is made to the fact that the description of the invention and the exemplified embodiments are of course fundamentally not to be understood as being limiting in terms of a particular physical embodiment of the invention. All the features shown and explained in conjunction with individual embodiments of the invention can be provided in different combinations in the subject matter in accordance with the invention in order to simultaneously achieve the advantageous effects thereof. For example, it is thus likewise within the scope of the invention to provide other operating or control elements of the medical machine for inputting the confirmation input alternatively or cumulatively to the graphical user interface. For example, these can be manually operated operating elements, joysticks, a mouse, a conventional keyboard or the like. For a person skilled in the art it is particularly obvious that the invention can be used not only for dialysis machines but also for other medical-technology machines in which messages have to be acknowledged by a confirmation input. The messages can be error messages but also other messages in relation to a machine status.

Furthermore, the components of the medical system for message processing or error processing can be embodied separately on a plurality of physical products.

The scope of protection of the present invention is determined by the claims and is not limited by the features explained in the description or shown in the figures.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A dialysis machine, comprising a processor and a non-transitory computer-readable medium, the non-transitory computer-readable medium having processor-executable instructions stored thereon, wherein the processor-executable instructions, when executed by the processor, facilitate performance of the following:
outputting a number of consecutive machine error messages; and
determining a number of consecutive confirmation inputs received in response to the consecutive machine error messages as an ACTUAL value.

2. The dialysis machine according to claim 1, wherein the processor-executable instructions, when executed, further facilitate:
storing a preconfigured TARGET value;
comparing the ACTUAL value with the TARGET value; and
initiating a modified machine control in response to the ACTUAL value exceeding the TARGET value.

3. The dialysis machine according to claim 2, wherein the processor-executable instructions, when executed, further facilitate:
outputting the ACTUAL value and the TARGET value.

4. The dialysis machine according to claim 1, wherein the processor-executable instructions, when executed, further facilitate:
evaluating whether a currently output machine error message has been responded to with a confirmation input;
evaluating how often a currently output machine error message has been responded to with a confirmation input within a time frame; and/or
evaluating by which operating person a currently output machine error message was responded to with a confirmation input.

5. The dialysis machine according to claim 1, wherein the processor-executable instructions, when executed, further facilitate:
resetting the ACTUAL value in response to detecting a status change on the dialysis machine.

6. A method for operating a dialysis machine, comprising:
outputting, by the dialysis machine, a number of consecutive machine error messages; and
determining, by the dialysis machine, a number of consecutive confirmation inputs received in response to the consecutive machine error messages as an ACTUAL value.

7. The method according to claim 6, further comprising:
comparing the ACTUAL value a preconfigurable TARGET value; and
controlling, in response to the ACTUAL value exceeding the TARGET value, the dialysis machine in a modified manner by automatically outputting a modified message based on the ACTUAL value.

8. The method according to claim 7, wherein a plurality of TARGET values are configurable which define different value ranges, and wherein a different control procedure corresponds to each value range, and wherein controlling the dialysis machine in the modified manner depends on which value range the ACTUAL value is located in.

9. The method according to claim 6, wherein the machine error messages and/or the confirmation inputs are output or detected on a touchscreen monitor of the dialysis machine.

10. The method according to claim 6, wherein the ACTUAL value is automatically reset if it is detected that a status causing an output machine error message no longer exists.

11. The method according to claim 6, wherein a modified control of the dialysis machine is activated or deactivated after a predefinable event has occurred.

12. The method according to claim 6, wherein a number of confirmation inputs is stored and/or output for all output machine error messages itemized for each user.

13. The method according to claim 6, further comprising:
detecting a user identity; and
controlling the dialysis machine in a modified manner in response to the ACTUAL value and the user identity.

14. The method according to claim 6, wherein a TARGET value is configured based on the user identity.

15. A method for operating a dialysis machine, comprising:
determining, by the dialysis machine, an error status via sensors of the dialysis machine;
outputting, by the dialysis machine, a machine error message in response to the determined error status;
receiving, by the dialysis machine, confirmation input via a user interface of the dialysis machine and determining whether the error status has been addressed; and
based on the machine error message being output a predetermined consecutive number of times without the error status being addressed, initiating, by the dialysis machine, modified machine operation.

16. The method according to claim 15, wherein the modified machine operation comprises outputting of a modified machine error message.

17. The method according to claim 15, wherein the modified machine operation comprises outputting instructions for addressing the error status.

18. The method according to claim 15, wherein the modified machine operation comprises modifying the user interface of the dialysis machine.

19. The method according to claim 15, wherein before initiating the modified machine operation based on the machine error message being output a predetermined consecutive number of times, the method further comprises:
consecutively outputting the same machine error message each time a confirmation input is received without the error status being addressed.
